(19) United States
(12) Patent Application Publication
(10) Pub. No.: US 2014/0193446 A1
(43) Pub. Date: Jul. 10, 2014

(54) BIODEGRADABLE HIGH-EFFICIENCY DENGUE VACCINE, METHOD FOR MAKING THE SAME, AND PHARMACEUTICAL COMPOSITION COMPRISING THE SAME

(71) Applicant: National Cheng Kung University, Tainan City (TW)

(72) Inventors: YEE-SHIN LIN, TAINAN CITY (TW); YU-HUNG CHEN, TAINAN CITY (TW)

(21) Appl. No.: 13/924,793

(22) Filed: Jun. 24, 2013

(30) Foreign Application Priority Data
Jan. 9, 2013 (TW) 102100790

(51) Int. Cl. A61K 39/12

(52) U.S. Cl. CPC A61K 39/12
USPC 424/186.1

(57) ABSTRACT

The present invention is related to a biodegradable high-efficiency dengue vaccine, a method for making the same, and a pharmaceutical composition comprising the same. The biodegradable high-efficiency dengue vaccine comprises a biodegradable nanocomplex with electric properties holding a dengue viral protein inside. An organism has antibody responses after vaccination with the biodegradable nanocomplex twice. Accordingly, in comparison with the Alum adjuvant and Ribi adjuvant used in the traditional dengue vaccine of the prior art, the vaccination times in the present invention is decreased to further reduce the vaccination cost, so the biodegradable high-efficiency dengue vaccine is good for being a commercial vaccine.

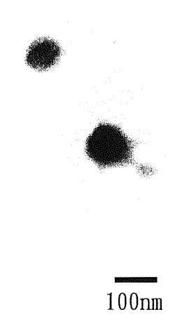

### BACKGROUND OF THE INVENTION

1. Field of the Invention

The present invention relates a biodegradable high-efficiency dengue vaccine. More particularly, a biodegradable nanocomplex with electric property holds a dengue viral protein inside, after vaccination with the foregoing biodegradable nanocomplex twice, an organism has an antibody response, and the antibody production is increased widely for inducing immune responses efficiently.

2. Description of Related Art

Dengue fever, also known as breakbone fever, is an acute viral disease transmitted by Adeds aegypti or Aedes albopictus, and its symptoms include fever (39° C.-40° C.) or aversion to cold, skin rash with fatigue in limb, muscle pains, frontal headache, orbital pain, abdominal pain, backache (i.e. the origin of the term “breakbone fever”), sore throat, and maybe vomiting, fainting, etc. The commonly mentioned dengue fever is classic dengue fever, also called as primary dengue fever. In addition, severe and life-threatening dengue fever characterized by hemorrhage or shock may be developed, also called dengue hemorrhagic fever (DHF) or dengue shock syndrome (DSS), or secondary dengue fever. It is estimated that there are about 50 million to 100 million cases of dengue infection worldwide each year, with about 250,000 to 500,000 cases of dengue hemorrhagic fever. Hence, the prevention and treatment of dengue fever is an important issue for the governments of many countries. Since dengue virus is the major pathogen of dengue disease, the early detection or prevention with effective vaccine can efficiently control morbidity and death rates of dengue fever.

Please refer to the Taiwan Patent Publication No. 201210614 “Dengue vaccine, medicinal composition comprising the same, and nucleotide sequence” and the Taiwan Patent Publication No. 201210615 “Dengue vaccine, medicinal composition comprising the same, nucleotide sequence, and antibody composition”, which are applied by the inventors of the present invention. A dengue vaccine causing no autoimmunity to avoid the cross-reaction between endothelial cell and platelets and being able to shorten the bleeding time is disclosed in the foregoing applications, the contents of which are hereby incorporated by reference herein. The foregoing dengue vaccine has prospective effect in actual operation, but there is deficiency due to the aluminum hydroxide gel (also called as immunostimulant) as an adjuvant in the foregoing dengue vaccine. The ability of the aluminum hydroxide is undesired to enhance the immune response in the organism.

Commonly, the adjuvant action mechanism generally comprises: (a) increasing the life or the immunity of an antigen in the vaccine, (b) delivering antigen to the antigen-presenting cell, (c) improving antigen display in antigen-presenting cell, and (d) inducing the production of immunoregulatory cytokine. The mineral adjuvant is one of the common adjuvant, such as metal salts of zinc, calcium, cerium, chromium, iron, and beryllium. The aluminum salts, such as aluminum hydroxide and aluminum phosphate, is the most commonly used, and is also called as Alum adjuvant. The mechanism of the Alum adjuvant refers to the antigen being absorbed on the aluminum salt, which is also used as the immunostimulant, and when the antigen is taken up by antigen-presenting cell, the immunostimulant absorbed by the antigen stimulates the antigen-presenting cell at the same time.

Please refer to the U.S. Pa. No. 7,357,963. It disclosed a process for the manufacture of a vaccine, in which an adjuvant composition containing an immunostimulant adsorbed onto a first metallic salt particle substantially free of antigen is mixed with an antigen adsorbed onto a second metallic salt particle. The antigen is derived from human immunodeficiency virus, varicella zoster virus, human cytomegalovirus, dengue virus, hepatitis A, B, C or E virus. Actually, the Alum adjuvant is applied to over 50% of the commercial vaccine product, including hepatitis B vaccine (Alum-HBsAg), diphtheria and tetanus toxoid vaccine (Alum-DT), etc. The foregoing antigen-metal complex vaccine is used for years and it is proved that the complex is absorbed easily by the antigen-presenting cell, but it is doubted that the safety of the heavy metal. Accordingly, the safety adjuvant used in the vaccine application should be developed to avoid the unsafe problems resulting from the Alum adjuvant and to enhance the antibody production for better immune responses and lowering the vaccination times and the cost.

### SUMMARY OF THE INVENTION

In view of the foregoing disadvantages of the traditional dengue vaccine in actual operation, the object of the present invention is to provide a biodegradable nanocomplex with electric property holding a dengue viral protein inside. After vaccination with the biodegradable nanocomplex twice, an organism has antibody responses and the antibody production is widely increased, so that the vaccine induces the immune responses efficiently.

Accordingly, a biodegradable high-efficiency dengue vaccine is provided, which comprises a biodegradable nanocomplex with electric property holding a dengue viral protein inside. After vaccination with the biodegradable nanocomplex twice, an organism has antibody responses. In comparison with the Alum adjuvant and Ribi adjuvant used in the traditional dengue vaccine of the prior art, the vaccination times of the biodegradable high-efficiency dengue vaccine in the present invention is decreased to reduce the vaccination cost, so the biodegradable high-efficiency dengue vaccine is good for being a commercial vaccine. In addition, because of the biodegradability of the nanocomplex, the dengue vaccine is decomposed, absorbed and removed easily and naturally by the human body after it enters the human body. It resolves the unsafe problem resulting from the heavy metal of the Alum adjuvant, and the dengue viral protein held in the biodegradable nanocomplex is released slowly for the sustained release.

According to an embodiment of the present invention, the dengue viral protein comprises a nonstructural chimeric protein DJ NS1. The nonstructural chimeric protein DJ NS1 comprises N-terminal amino acid 1-270 of a dengue virus nonstructural protein (DV NS1) and C-terminal amino acid 271-352 of a Japanese encephalitis virus nonstructural protein (JEV NS1). Moreover, the nonstructural chimeric protein DJ NS1 has more than 90%, even more than 95%, sequence similarity to the SEQ. ID. NO. 1.

According to another embodiment of the present invention, the dose of the biodegradable nanocomplex is no more than 25 μg for the first vaccination. Moreover, when the biodegradable nanocomplex is made from the chitosan with positive charge and the polyglutamic acid with negative charge, after the second vaccination with the dengue vaccine, the organism has an antibody titer of 1:256000. In addition, when the biodegradable nanocomplex is made from the chitosan with positive charge and the heparin with negative charge, after the second vaccination with the dengue vaccine, the organism has an antibody titer of 1:32000 at least. Accordingly, in comparison with the prior art, the dengue vaccine of the present invention sharply increases the antibody production to induce the immune responses efficiently for enhancing the protection effect of the vaccine.

A method for making a biodegradable high-efficiency dengue vaccine is also provided. The method comprises the following steps. A first solution comprising a first biodegradable macromolecule with a first electric property is prepared. According to an embodiment, the first biodegradable macromolecule is heparin or polyglutamic acid. Next, a dengue viral protein with the same electric property as the first biodegradable macromolecule is dissolved in the first solution to be a mixture solution with the first electric property. Then, a second solution comprising a second biodegradable macromolecule with a second electric property is prepared. The first electric property is opposite to the second electric property. According to an embodiment of the present invention, the second biodegradable macromolecule is chitosan or collagen. Finally, the mixture solution is added into the second solution for forming a biodegradable nanocomplex by attraction force between the different electric properties, and the biodegradable nanocomplex holds the dengue viral protein inside.

A pharmaceutical composition comprising the foregoing biodegradable high-efficiency dengue vaccine is also provided. The pharmaceutical composition is used for producing a vaccine or a drug for treating or preventing hemorrhagic dengue fever or dengue shock syndrome.

### BRIEF DESCRIPTION OF THE DRAWINGS

The FIGURE is an electron microscope image of a biodegradable nanocomplex holding a dengue viral protein inside according to an embodiment of the present invention.

### DETAILED DESCRIPTION OF THE PREFERRED EMBODIMENT

A biodegradable high-efficiency dengue vaccine is disclosed, which comprises a biodegradable nanocomplex with electric properties holding a dengue viral protein inside. After vaccination with the biodegradable nanocomplex twice, an organism has antibody responses. The dose of the biodegradable nanocomplex is no more than 25 μG for the first vaccination. It is noted that the foregoing dengue viral protein can be dengue envelope protein or dengue nonstructural protein. According to an embodiment of the present invention, the dengue viral protein is disclosed in the Taiwan Patent Publication No. 201210614 “ Dengue vaccine, medicinal composition comprising the same, and nucleotide sequence”, the contents of which are hereby incorporated by reference herein. The dengue viral protein comprises a nonstructural chimeric protein DJ NS1. The nonstructural chimeric protein DJ NS1 comprises N-terminal amino acid 1-270 of a dengue virus nonstructural protein (DV NS1) and C-terminal amino acid 271-352 of a Japanese encephalitis virus nonstructural protein (JEV NS1). Moreover, the sequence similarity between the nonstructural chimeric protein DJ NS1 and the SEQ. ID. NO.1 is more than 90%, even more than 95%.

The method for making a biodegradable high-efficiency dengue vaccine of the present invention, which allows an organism to have an antigen tilter of 1:256000 after the second vaccination, could be performed by the following examples to further show the range of the actual application, but not to limit the spirit of the present invention:

### EXAMPLE 1

### Prepare a First Solution Comprising a First Biodegradable Macromolecule

The first solution comprises a first biodegradable macromolecule with first electric property, and the first biodegradable macromolecule is polyglutamic acid (γ-PGA) or heparin and the first electric property is negative charge, for example. In detail, a proper amount of polyglutamic acid was added into de-ionized water and stirred by electromagnetic stirrer until the polyglutamic acid was totally dissolved. Then, the sodium in the polyglutamic acid solution was removed by membrane dialysis. The dialysis process was accomplished at 4° C. for preventing bacterial growth. After dialysis, the polyglutamic acid solution was put at −20° C. for being totally frozen. Then, the water content of the frozen polyglutamic acid solution was removed by lyophilization to obtain the crystallized powder of the polyglutamic acid. The crystallized powder of the polyglutamic acid was stored in a sterilized tube and put in a moisture-proof box. Finally, a proper amount of crystallized powder of the polyglutamic acid was taken and dissolved in the de-ionized water in a desired concentration, which was the first solution comprising the first biodegradable macromolecule with negative charge.

However, one skilled in the art will readily recognize that the foregoing method for preparation of the first solution is one of embodiments. After reading and understanding the descriptions of the present invention, it will be obvious to those skilled in the art that various modifications may be made and not limited to the foregoing embodiment.

### EXAMPLE 2

### Prepare a mixture Solution Containing a Dengue Viral Protein and the First Solution

A dengue viral protein with the same electric property as the first biodegradable macromolecule was dissolved in the first solution to form a mixture solution with negative charge. The dengue viral protein was disclosed in the Taiwan Patent Publication No. 201210614 “Dengue vaccine, medicinal composition comprising the same, and nucleotide sequence.” The dose of the dengue viral protein was 100 □μg, 200 □μg, or 400 μg, and it is not limited here.

### EXAMPLE 3

### Prepare a Second Solution Comprising a Second Biodegradable Macromolecule

A second solution comprising a second biodegradable macromolecule with a second electric property is prepared, and the first electric property is opposite to the second electric property. The second biodegradable macromolecule is chitosan or collagen, for example, and the second electric property is positive charge. In detail, the second biodegradable macromolecule was chitosan. 5g low-viscous chitosan was added into 495 ml de-ionized water with 5 ml glacial acetic acid and stirred by electromagnetic stirrer until the chitosan solution stays in a yellow and pellucid state. Next, the glacial acetic acid in the chitosan solution was removed by membrane dialysis, and the pH of the chitosan solution was about 6.5 after dialysis. Then, the chitosan solution was filtrated by air suction filter to remove the impurity. Finally, the chitosan solution was heated and stirred at 135 ° C. for being concentrated until the concentration of the chitosan solution reaches 20-30 mg/ml, which was the second solution comprising the second biodegradable macromolecule with positive charge.

Similarly, one skilled in the art will readily recognize that the foregoing method of preparation of the second solution is one of embodiments. After reading and understanding the descriptions of the present invention, it will be obvious to those skilled in the art that various modifications may be made and not limited to the foregoing embodiment.

It is noted that the foregoing first and second biodegradable macromolecule can be natural macromolecules, such as heparin or polyglutamic acid, and chitosan or collgen, respectively. The foregoing first and second biodegradable macromolecule also can be artificially biodegradable macromolecules.

### EXAMPLE 4

### Form a Biodegradable Nanocomplex

The mixture solution was added into the second solution to form a biodegradable nanocomplex by attraction force between the different electric properties, and the dengue viral protein was held in the biodegradable nanocomplex. The FIGURE is an electron microscope image of a biodegradable nanocomplex holding a dengue viral protein inside according to an embodiment of the present invention. It is worth noted that the dengue viral protein with negative charge is mixed with the polyglutamic acid solution with negative charge to form a mixture solution first, and then the mixture solution is mixed with the chitosan solution with positive charge to form the biodegradable nanocomplex solution, in which the structure of the biodegradable nanocomplex is more stable. However, the dengue viral protein with negative charge also can be mixed with the chitosan solution with positive charge first to form a mixture solution, and then the mixture solution is mixed with the polyglutamic acid solution. Because of the biodegradability of the first and second macromolecule, the nanocomplex formed from the first and second macromolecule has biodegradability as well. The biodegradability means that the nanocomplex is decomposed, absorbed and removed easily and naturally by the human body after it enters the human body, and the dengue viral protein held in the nanocomplex is released slowly for the sustained release. Table 1 is the particle size and the zeta potential of the biodegradable nanocomplex with and without holding the dengue viral protein. The charge ratio of the chitosan with positive charge to the polyglutamic acid with negative charge is preferably 4:1 because the structure of the biodegradable nanocomplex stays in a most stable state under this condition. The results of the particle size and zeta potential are the average value of three biodegradable nanocomplexes.

TABLE 1

| | without holding | with holding |
|---|---|---|
| | dengue viral protien | dengue viral protein |
| Size (nm) | 126.4 ± 5.1 | 124.5 ± 1.8 |
| Zeta potential (mV) | 83.5 ± 6.2 | 15.4 ± 0.7 |

### EXAMPLE 5

### Vaccination with the Biodegradable Nanocomplex Holding Dengue Viral Protein Inside on a Mouse Model

Mice are vaccinated with the biodegradable nanocomplex holding the dengue viral protein inside as a model compound. C3H/HeN mice were obtained from the Jackson Laboratory, and maintained on standard laboratory food and water in the Laboratory Animal Center of National Cheng Kung University Medical College in Taiwan (R.O.C.). Housing, breeding, and experimental use of the animals were performed in strict accordance with the Experimental Animal Committee in the laboratory animal center of National Cheng Kung University. Table 2 is the results of a titer of a neutralizing antibody in the mice vaccinated by the biodegradable nanocomplex holding the dengue viral protein inside of the present invention, by the traditional Alum adjuvant, or by the traditional Ribi adjuvant.

TABLE 2

| | Antibody titer to the DJ NS1(×10³) | | | | | |
|---|---|---|---|---|---|---|
| | Nanocomplex | | Alum | | Ribi | |
| Vaccination | (μg/mouse) | | (μg/mouse) | | (μg/mouse) | |
| time | 25 | 50 | 25 | 50 | 25 | 50 |
| First | ND | ND | ND | ND | ND | ND |
| Second | 2⁸ | 2⁹ | ND | ND | ND | ND |
| Third | 2¹⁰ | 2¹¹ | 2⁶ | ₂8 | 2⁷ | 2⁸ |

According to Table 2, after vaccination twice, a specific antibody response was induced by the dengue vaccine comprising the biodegradable nanocomplex of the present invention, and the mice had the antibody titer of 1:256000 when the dose of the biodegradable nanocomplex in the dengue vaccine is 25 μg per vaccination. Accordingly, the vaccination times of the biodegradable high-efficiency dengue vaccine in the present invention is decreased, so the biodegradable high-efficiency dengue vaccine is good for being a commercial vaccine. The ND means that the antibody titer is non-detectable. The antibody titer was measured by an ELISA standard protocol. The time of antibody response induced by the biodegradable nanocomplex was faster than that induced by the traditional Alum adjuvant and Ribi adjuvant. In detail, the traditional Alum adjuvant and Ribi adjuvant induced the specific antibody response to the dengue viral protein in the mice until the third vaccination. Moreover, after the third vaccination, the antibody titer induced by the biodegradable nanocomplex of the present invention was higher than that induced by the foregoing Alum adjuvant and Ribi adjuvant. It is suggested that the biodegradable nanocomplex holding dengue viral protein inside enhanced the adjuvant effect in the dengue vaccine. The foregoing Ribi adjuvant was non-toxic and non-immunity oil-in-water emulsions in Ribi adjuvant system (RAS) developed by the Ribi Immunochem Research Inc. in 1985.

In the other embodiment of the present invention, the biodegradable nanocomplex made from heparin as the first biodegradable macromolecule and chitosan as the second biodegradable macromolecule also induced the specific antibody response to the dengue viral protein in mice after the second vaccination, and the dose of the biodegradable nanocomplex in the dengue vaccine is 25 μg per vaccination. The organism had the antibody titer of 1:32000 at least after the second vaccination.

A pharmaceutical composition comprising the dengue vaccine comprising the foregoing biodegradable nanocomplex is also provided, which is used for producing a vaccine or a drug for treating or preventing hemorrhagic dengue fever or dengue shock syndrome. The pharmaceutical composition comprises the foregoing biodegradable high-efficiency dengue vaccine or an addition salts thereof with a pharmaceutically acceptable base, and at least one pharmaceutically acceptable excipient. Moreover, the pharmaceutical composition of the present invention can be administered to animals in any existing ways, i.e. oral, nasal, mucosal, topical, dermal, and parenteral administration, wherein parenteral administration is intravenous, intraperitoneal, intradermal, subcutaneous, or intramuscular administration. The pharmaceutical composition of the present invention also can be administered via the combination of the foregoing administrations. For example, the first vaccination is via parenteral administration, and the second vaccination is via mucosal administration. In addition, the dose of the pharmaceutical composition varies depending on the species, age, weight, and status of individuals, the disease to be prevented or treated, the seriousness of the disease, the specific compound use in the pharmaceutical composition, and administration methods. One skilled in the art will readily recognize the publication content of the present invention, a proper dose can be decided by the routine experiment, and after the first vaccination, the organism can be decided to receive one or more additional vaccinations at a proper interval.

According to the above description, in comparison with the traditional technique, the biodegradable high-efficiency dengue vaccine, the method for making the same and the pharmaceutical composition of the same according to the present invention has the advantages as following:
1. The dengue vaccine comprising the biodegradable nanocomplex of the present invention can induce a specific antibody response to the dengue viral protein in mice after vaccination twice. In comparison with the Alum adjuvant and Ribi adjuvant used in the traditional dengue vaccine of the prior art, the vaccination times of the biodegradable high-efficiency dengue vaccine in the present invention is decreased to further reduce the vaccination cost, so the biodegradable high-efficiency dengue vaccine is good for being a commercial vaccine.
2. After vaccination with the dengue vaccine of the present invention twice, the organism has the antibody titer of 32000 at least. In comparison with the prior art, the dengue vaccine of the present invention substantially increases the antibody production to induce the immune response efficiently for enhancing the protection effect of the vaccine.
3. The biodegradable nanocomplex of the dengue vaccine of the present invention is made from the mix of the biodegradable polyglutamic acid (or heparin) and chitosan to hold the dengue viral protein inside. Accordingly, the dengue vaccine is decomposed, absorbed and removed easily and naturally by the human body after it enters the human body. It resolves the unsafe problems resulting from the heavy metal of the Alum adjuvant, and the dengue viral protein held in the nanocomplex is released slowly for the sustained release.

What is claimed is:

1. A biodegradable high-efficiency dengue vaccine, comprising:
a biodegradable nanocomplex with an electric property holding a dengue viral protein inside, wherein an organism has an antibody response after vaccination with the biodegradable nanocomplex twice.

2. The biodegradable high-efficiency dengue vaccine as claimed in claim 1, wherein the dose of the biodegradable nanocomplex is no more than 25 μg for a first vaccination.

3. The biodegradable high-efficiency dengue vaccine as claimed in claim 1, wherein the organism has an antibody titer of 32000 at least after a second vaccination with the biodegradable nanocomplex.

4. The biodegradable high-efficiency dengue vaccine as claimed in claim 3, wherein the organism has the antibody titer of 1:256000 after the second vaccination with the biodegradable nanocomplex.

5. The biodegradable high-efficiency dengue vaccine as claimed in claim 1, wherein the dengue viral protein comprising:
a nonstructural chimeric protein DJ NS1, comprising:
a N-terminal amino acid 1-270 of a dengue virus nonstructural protein (DV NS1); and
a C-terminal amino acid 271-352 of a Japanese encephalitis virus nonstructural protein (JEV NS1).

6. The biodegradable high-efficiency dengue vaccine as claimed in claim 5, wherein the sequence similarity between the nonstructural chimeric protein DJ NS1 and the SEQ. ID. NO.1 is more than 90%.

7. The biodegradable high-efficiency dengue vaccine as claimed in claim 1, wherein the biodegradable nanocomplex is made from a chitosan with positive charge and a heparin or polyglutamic acid with negative charge.

8. A method for making a biodegradable high-efficiency dengue vaccine, comprising the steps of:
preparing a first solution comprising a first biodegradable macromolecule with a first electric property;
dissolving a dengue viral protein with the same electric property as the first biodegradable macromolecule into the first solution to be a mixture solution with the first electric property;
preparing a second solution comprising a second biodegradable macromolecule with a second electric property, wherein the first electric property is opposite to the second electric property; and
adding the mixture solution into the second solution for forming a biodegradable nanocomplex by attraction force between the different electric properties, wherein the dengue viral protein is held in the biodegradable nanocomplex.

9. The making method as claimed in claim 8, wherein the first macromolecule is heparin or polyglutamic acid, and the first electric property is a negative charge.

10. The making method as claimed in claim 8, wherein the second macromolecule is chitosan or collagen, and the second electric property is a positive charge.

11. The making method as claimed in claim 8, wherein the dengue viral protein comprising:
a nonstructural chimeric protein DJ NS1, comprising:
a N-terminal amino acid 1-270 of a dengue virus nonstructural protein (DV NS 1); and
a C-terminal amino acid 271-352 of a Japanese encephalitis virus nonstructural protein (JEV NS1).

12. The making method as claimed in claim 11, wherein the sequence similarity between the nonstructural chimeric protein DJ NS1 and the SEQ. ID. NO.1 is more than 90%.

13. A pharmaceutical composition comprising:
a biodegradable high-efficiency dengue vaccine as claimed in claim 1 or an addition salt thereof with a pharmaceutically acceptable base; and
at least one pharmaceutically acceptable excipient.

14. The pharmaceutical composition as claimed in claim 13. which is used for producing a vaccine or a drug for treating or preventing hemorrhagic dengue fever or dengue shock syndrome.
